(19) United States
(12) Patent Application Publication
(10) Pub. No.: US 2026/0351419 A1
(43) Pub. Date: Nov. 5, 2026

(54) Method and system for providing and/or assessing a patient’s medical condition

(71) Applicant: BIOTRONIK SE & Co. KG, Berlin (DE)

(72) Inventors: Hendrik DEUTSCHMANN, Berlin (DE); Jens MUELLER, Berlin (DE); Daniel KRUEGER, Bestensee (DE); Ha Uyen DO, Berlin (DE); Richard JORDAN, Berlin (DE); Marc VOSKAMP, Kleinmachnow (DE)

(73) Assignee: BIOTRONIK SE & Co. KG, Berlin (DE)

(21) Appl. No.: 19/118,709

(22) PCT Filed: Sep. 26, 2023
(86) PCT No.: PCT/EP2023/076460

§ 371 Date: Apr. 4, 2025

(30) Foreign Application Priority Data
Oct. 17, 2022 (EP) 22201921.8
Apr. 4, 2023 (EP) 23166511.8

(51) Int. Cl. G16H 50/30; G16H 10/20; G16H 10/60; G16H 40/67

(52) U.S. Cl. CPC G16H 50/30; G16H 10/20; G16H 10/60; G16H 40/67

(57) ABSTRACT

A computer-implemented method for assessing a patient's medical condition, including the steps of receiving patient medical data including a plurality of medical parameters acquired by an implantable medical device and/or wearable medical device and applying an algorithm to the received patient medical data, wherein if at least one predetermined condition is fulfilled, triggering a patient information request.

### CROSS-REFERENCE TO RELATED APPLICATIONS

This application is the United States National Phase under 35 U.S.C. § 371 of PCT International Patent Application No. PCT/EP2023/076460, filed on Sep. 26, 2023, which claims the benefit of European Patent Application No. 23166511.8, filed on Apr. 4, 2023, and European Patent Application No. 22201921.8, filed on Oct. 17, 2022, the disclosures of which are hereby incorporated by reference herein in their entireties.

### TECHNICAL FIELD

The invention relates to a computer-implemented method for providing and/or assessing a patient's medical condition.

Furthermore, the invention relates to a system for assessing a patient's medical condition.

### BACKGROUND

In the field of disease management questionnaires are generally either sent out manually via mail or collected verbally via a phone call by a health care provider when more information from the patient is required in order to track and evaluate the health condition of the patient. Collecting this information and/or triggering a transmission of the questionnaire requires action to be taken by the health care provider.

Patient follow-up can be time-consuming for the health care provider since it is not always possible to reach patients. Moreover, patients are often annoyed by frequent calls of the health care provider and being asked the same questions when they are feeling fine.

In view of this, the process of contacting patients has more recently been replaced by triggering a questionnaire that is sent to a patient communication device in reaction to at least one of a plurality of medical parameters.

It is therefore an object of the present invention to provide an improved method for providing and/or assessing a patient's medical condition capable of more quickly and/or accurately evaluating and/or classifying said patient's medical condition.

The present disclosure is directed toward overcoming one or more of the above-mentioned problems, though not necessarily limited to embodiments that do.

### SUMMARY

The object is solved by a computer implemented method for providing and/or assessing a patient's medical condition having the features of claim 1.

Furthermore, the object is solved by a system for providing and/or assessing a patient's medical condition having the features of claim 13.

Moreover, the object is solved by a computer program having the features of claim 14 and a computer-readable data carrier having the features of claim 15.

Further developments and advantageous embodiments are defined in the dependent claims.

The present invention provides a computer implemented method for providing and/or assessing a patient's medical condition. The method comprises receiving patient medical data comprising a plurality of medical parameters acquired by an implantable medical device and/or wearable medical device and/or receiving patient medication data. In addition to the wearable medical device or instead of the wearable medical device, another wearable device may be used, such as a smart watch, a sports watch, a fitness tracker and/or a pedometer.

Furthermore, the method comprises applying an algorithm to the received patient medical data and/or the received patient medication data, wherein if at least one predetermined condition is fulfilled, triggering a patient information request.

According to a further aspect of the invention, applying the algorithm to the received patient medical data and/or the received patient medication data comprises, wherein if at least one of the plurality of medical parameters deviates from a predetermined normal range, triggering a patient information request, wherein a type of patient information request triggered depends on which of the at least one of the plurality of medical parameters deviates from the predetermined normal range.

Thus, the at least one predefined condition may be that at least one of the plurality of medical parameters deviates from the predetermined normal range.

The algorithm may comprise presenting and/or showing of the received patient medical data and/or plurality of medical parameters to a physician or a health care provider (HCP). The presenting and/or showing (of the medical data and/or parameter) may include indications (e.g. by highlighting) of a worsening of the patient's health condition. Subsequently, the physician or HCP may decide whether the at least one condition is fulfilled and return corresponding information to the algorithm (e.g. by entering the information into a graphical user interface). Depending on whether the predefined condition is fulfilled, the patient information request is then triggered or not.

Additionally or alternatively, the algorithm may comprise (automatically or manually) comparing values extracted from the received medical data and/or the plurality of medical parameters with at least one predefined threshold value, wherein the predetermined condition is fulfilled if a predetermined amount of extracted values (e.g. three values extracted from the received medical data and/or parameter) exceeds or crosses the at least one predefined threshold value. As already mentioned, triggering of the patient information request occurs, if the at least one predetermined condition is fulfilled.

Additionally or alternatively, the comparing (of extracted values) may comprise adding at least two of the extracted values to a sum of values and comparing this sum of values with the predefined threshold value, wherein the predetermined condition is fulfilled if the sum values (e.g. sum of three extracted values) exceeds or crosses the at least one predefined threshold value.

In addition, the present invention provides a system for providing and/or assessing a patient's medical condition. The system comprises a data storage device configured to receive patient medical data comprising a plurality of medical parameters acquired by an implantable medical device and/or wearable medical device and/or configured to receive patient medication data.

Moreover, the system comprises a computing device configured to apply an algorithm to the received patient medical data and/or the received patient medication data, wherein if at least one predetermined condition is fulfilled, the computing device is further configured to trigger a patient information request.

According to a further aspect of the invention, the computing device is further configured to apply the algorithm to the received patient medical data, wherein if at least one of the plurality of medical parameters deviates from a predetermined normal range, the computing device is further configured to trigger a patient information request, wherein a type of patient information request triggered depends on which of the at least one of the plurality of medical parameters deviates from the predetermined normal range.

The algorithm preferably operates on a server of an IT service provider, wherein the patient medical data is transmitted from the implantable medical device to the server of the IT service provider via a relay device arranged within a communication range of the implantable medical device. The implantable medical device communicates with the relay device ordinarily via coil-based communication and/or mics-band radio.

Alternatively, or in addition, the implantable medical device may communicate with the server of the IT service provider via a patient communication device, in particular a smartphone and/or tablet computing device via e.g. Bluetooth Low Energy (BLE) communication.

Furthermore, the present invention provides a computer program with program code to perform the method of the present invention when the computer program is executed on a computer. In addition, the present invention provides a computer-readable data carrier containing program code of a computer program for performing the method of the present invention when the computer program is executed on a computer.

The medical parameters are e.g. arrhythmia data, a heart rate, a patient activity, a chest impedance, and/or readings from (or measurements by) electrodes (or sensors) of the implantable medical device.

An idea of the present invention is to facilitate the process of triggering and evaluating a patient questionnaire/survey. With this trigger a disease specific survey/questionnaire is sent to the patient's communication device via an app. After the patient answers the questions displayed in the questionnaire the responses are sent back to the system, where it is accessible to the health care provider for further evaluation. The invention thus provides personalized care, more patient involvement and enhanced program adjustments based on additional patient data.

According to an aspect of the invention, triggering the patient information request comprises sending the patient information request, in particular a patient questionnaire, from a server of an IT service provider to a patient communication device, in particular a smart phone and/or tablet computing device, wherein the patient information request is accessible via a first front-end application on the patient communication device.

Thereby, the patient information request is conveniently accessible to the patient and can be answered at any time. By sending the patient information request from the server of the IT service provider to the patient communication device, a suitable one of the plurality of possible patient information requests stored on the server can be chosen to be transmitted to the patient communication device.

According to a further aspect of the invention, triggering the patient information request comprises sending a command from a server of an IT service provider to a patient communication device, in particular a smart phone and/or tablet computing device, to access the patient information request, in particular a patient questionnaire, By only sending the command from the server of the IT service provider to the patient communication device, one of a plurality of pre-installed patient information requests related to a patient medical condition can be invoked on the patient communication device.

The command and/or the patient information request is sent from the server of the IT service provider to the patient communication device using a remote network service operating on the server of the IT service provider. An app running on the patient communication device in this context is configured to connect to the remote network service in order to receive the command to access the patient information request and/or to receive the patient information request itself.

According to a further aspect of the invention, the patient medical data and/or the received patient medication data is received by a remote network service, in particular operating on the server of the IT service provider, and wherein the patient information request is sent to an app running on the patient communication device instantly or with a predetermined time delay.

The patient medication data comprises a type, interval and quantity of medication intake by the patient. Sending the patient communication device instantly provides the advantage that the patient is notified of the event and can thus contribute to providing the physician/health care provider with timely information by completing the questionnaire.

Sending the patient communication device with a predetermined time delay can be advantageous in particular if the event occurs during a patients sleep. In such a case sending the patient information request when the patient is awake avoids unnecessarily disturbing the patient.

According to a further aspect of the invention, the app running on the patient communication device compares a timestamp of the patient medical data, in particular a timestamp of an event triggering the patient information request, said timestamp being comprised by the patient information request, with calendar data of the patient communication device, wherein if a calendar entry is identified which matches and/or overlaps with the timestamp, a first predefined action is taken. Comparing a timing of the event with a time and date of a calendar entry enables considering said calendar data when determining a cause of the event.

According to a further aspect of the invention, the calendar entry is at least one of a sports activity, a meeting, a doctor's appointment and sleep, wherein the first predefined action is to add a further information request, in particular a question, to the patient information request, said further information request requesting whether the calendar entry matching and/or overlapping with the timestamp has had a physiological effect on the patient, in particular whether the calendar entry may be related to the event triggering the patient information request. The patient in answering the questions can thus provide additional information for the physician/health care provider that may be helpful in assessing the cause of the event.

According to a further aspect of the invention, the app running on the patient communication device compares a timestamp of the patient medical data, in particular a timestamp of an event triggering the patient information request, said timestamp being comprised by the patient information request, with physiological data acquired by the wearable medical device, said wearable medical device being at least one of a smart watch and a fitness tracker, wherein if the physiological data acquired by the wearable medical device deviates from a predefined normal range at the time of the timestamp, a second predefined action is taken. Considering the additional data acquired by the wearable medical device can useful information on the cause of the event.

According to a further aspect of the invention, the second predefined action is to add a further information request, in particular a question, to the patient information request, said further information request requesting whether the physiological data acquired by the wearable medical device deviating from the predefined normal range at the time of the timestamp has had a physiological effect on the patient, in particular whether said physiological data may be related to the event triggering the patient information request.

According to a further aspect of the invention, if the at least one of the plurality of medical parameters deviates from the predetermined normal range, an alarm and/or a notification to a health care provider is triggered, wherein said alarm and/or a notification to the health care provider is accessible via a second front-end application on a communication device, in particular a smart phone and/or a personal computer, of the health care provider.

Alternatively or in addition, whenever an event is found in the calendar at the timestamp, an additional, generic question could be added to the questionnaire, e.g. “Does or did the calendar entry x (with x for the title of the calendar entry) have or had an effect on your well-being or state of mind?”. Furthermore, if the event in the calendar additionally belongs to a specific category (e.g., “Sport” or “Meeting”), other additional detail questions could be added, e.g., “If you did sport, how intense was the session/sport?” or “If you had a meeting, did you have something (very) important to present?”

The healthcare provider is thus advantageously informed about a current medical condition of the patient and is aware of the fact that a patient information request has been triggered. Depending on the received patient medical data comprising the plurality of medical parameters acquired by the implantable medical device and/or the wearable medical device an alert level can be set, wherein based on the set alert level a further notification can be generated that is accessible via the second front-end application on the communication device, in particular the smart phone and/or the personal computer, of the health care provider.

According to a further aspect of the invention, the normal range of the plurality of medical parameters comprises a predetermined buffer range at an upper and/or lower bound of said normal range, wherein the alarm is maintained until the at least one of the plurality of medical parameters deviating from the predetermined normal range is within the normal range outside of the buffer range.

The provision of the buffer range at the upper and/or lower bound of said normal range enables that the alarm is canceled only if the medical condition of the patient is well within the normal range. Thus, a situation can be avoided, in which frequent patient information requests are triggered due to a medical parameter of the patient being close to a threshold value of the normal range.

According to a further aspect of the invention, the algorithm evaluates a response to the patient information request, wherein based on the response an alarm status is maintained or adjusted and/or a notification to the health care provider is generated comprising a recommended action. In doing so, based on frequent patient responses, and appropriate alarm status can be set and this information be transmitted to the healthcare provider for further evaluation. This way the interaction between healthcare provider and patient can be limited to essential follow-up.

According to a further aspect of the invention, if the response to the patient information request indicates that the patient is symptom free, a further patient information request is triggered after a predetermined time period.

Assuming that the alarm is still active after said predetermined time period that action thus assists the health care provider in determining whether or not the patient is continuously symptom free or may have developed symptoms after said predetermined time period.

According to a further aspect of the invention, if the alarm is still active after a predetermined time period after triggering the patient information request, a further patient information request is triggered after a further predetermined time period.

If the patient is in an alarm state for a longer period of time, he/she is naturally susceptible to deterioration of health condition. The regular receipt of questionnaires can thus provide the physician with indications as to whether the patient is subjectively experiencing an improvement, a worsening, or the occurrence or absence of symptoms.

According to a further aspect of the invention, the patient information request is related to an individual patient medical condition, wherein the type of patient information request triggered depends on a type of implant and/or wearable medical device of the patient, the medical data comprising the plurality of medical parameters acquired by the implant and/or wearable medical device of the patient and/or an electronic health record of the patient.

By basing the type of patient information request on the type of implant and/or wearable medical device of the patient, the medical data comprising the plurality of medical parameters acquired by the implant and/or wearable medical device of the patient and/or the electronic health record of the patient, the patient information request can thus advantageously be specifically directed to the medical condition of the patient due to the fact that patient data from possibly multiple sources is used to determine the content of the patient information request.

According to a further aspect of the invention, the algorithm outputs a health score, in particular a numerical value, of the patient, wherein the health score incorporates the patient medical data comprising the plurality of medical parameters acquired by the implantable medical device and/or wearable medical device having a first weight and the response to the patient information request having a second weight.

The health score output by the algorithm thus advantageously combines data measured by a patient medical device as well as feedback from the patient relating to his or her medical condition.

Of course, the plurality of medical parameters acquired by the implantable medical device and/or wearable medical device and the response to the patient information may also be displayed/outputted separately.

According to a further aspect of the invention, the first weight and/or the second weight is adjusted based on the response to the patient information request. The response to the patient information request thus advantageously serves to optimize the algorithm such that the health score output by the algorithm can more accurately reflect the medical condition of the patient.

According to a further aspect of the invention, if no response to the patient information request is received within a predetermined time period, a notification to the health care provider and/or a further patient information request is triggered. This advantageously frees up resources at the health care provider due to the fact that said task is not performed by medical personnel of the health care provider.

According to a further aspect of the invention, results of the patient information request, in particular the questionnaire, are transmitted back to the remote network service along with the data obtained from other sources such as the smart watch and/or the fitness tracker.

According to a further aspect of the invention, a sanity check is performed on the results of the patient information request, in particular on the answers to the questionnaire, wherein empty answers or conflicting answers are indicated, wherein the answers are presented to a physician / health care provider together with information on an original event triggering the patient information request and added data from other sources. The physician can in turn create a new program for the implantable medical device based on the received data.

According to a further aspect of the invention, the answers together with the original event information and the added data from other sources are automatically analyzed by the remote network service, wherein a program or program change is suggested to the physician. Based on this the physician can check and/or adjust the program of the implantable medical device.

If the patient decides not to answer, this state is also presented to the physician. Furthermore, the program is transmitted, in particular by the physician, as remote programming to the implantable medical device.

The herein described features of the computer-implemented method for assessing a patient's medical condition are also disclosed for the system for assessing a patient's medical condition and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The invention is explained in more detail below using exemplary embodiments, which are specified in the schematic figures of the drawings, in which:

FIG. 1 shows a flowchart of a computer-implemented method for assessing a patient's medical condition according to a preferred embodiment of the invention; and

FIG. 2 shows a diagram of a system for assessing a patient's medical condition according to the preferred embodiment of the invention.

### DETAILED DESCRIPTION

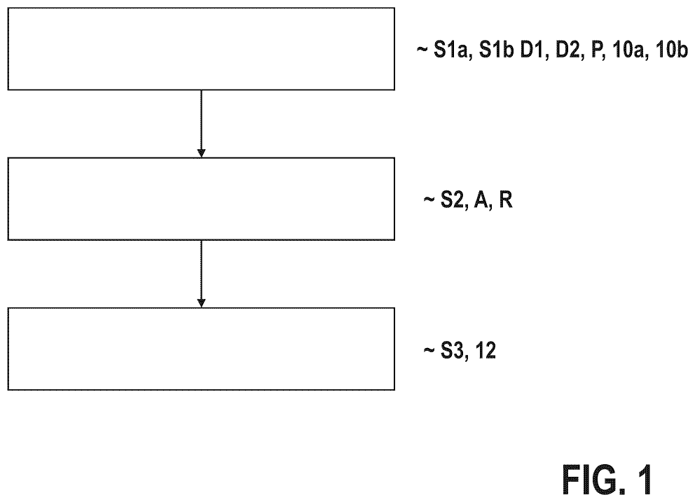

The method shown in FIG. 1 for providing and/or assessing a patient's medical condition comprises receiving Sla patient medical data DI comprising a plurality of medical parameters P acquired by an implantable medical device 10a and/or wearable medical device 10b, and/or receiving S1b patient medication data D2 acquired by a patient communication device 16. In addition to or instead of the wearable medical device 10b, another wearable device may be used for providing and/or assessing a patient's (medical) condition, such as a smart watch, a sports watch, a fitness tracker and/or a pedometer.

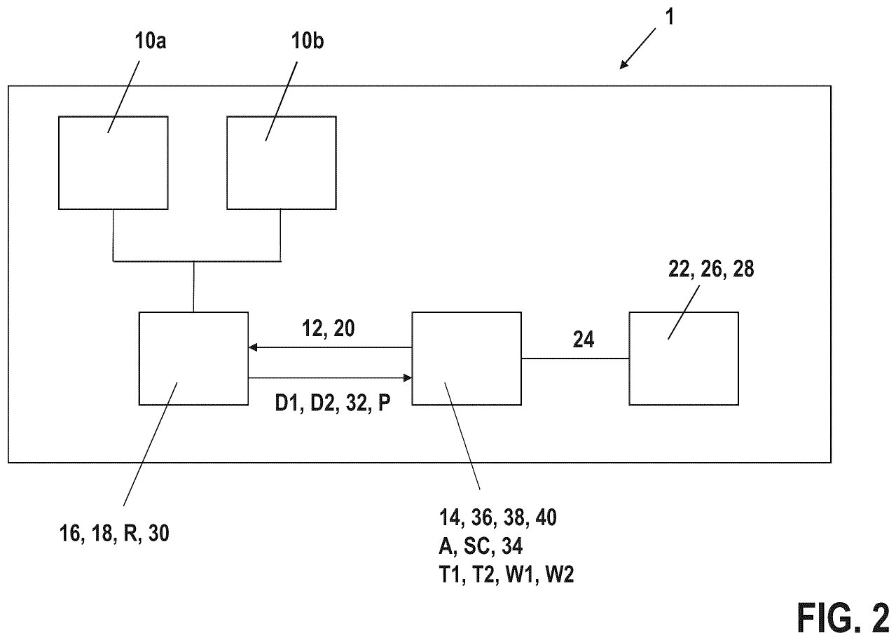

Receiving S1a patient medical data DI comprises sending/transmitting the plurality of medical parameters P from the implantable medical device 10a and/or the wearable medical device 10b via the patient communication device 16 to a server 14 (as shown in FIG. 2) and/or directly (or via another communication device different from the patient communication device 16) to a server 14 (as shown in FIG. 2).

Receiving S1b patient medication data D2 comprises sending/transmitting the medication data D2 from the patient communication device 16 to a server 14 (as shown in FIG. 2).

The method further comprises applying S2 an algorithm A to the received patient medical data D1 and/or the received patient medication data D2, wherein if at least one predetermined condition is fulfilled, triggering S3 a patient information request 12.

Once an event is transmitted from the implantable medical device 10a to a remote network service 40, an automated questionnaire will be prepared and transmitted to the patient communication device 16. The app on the patient communication device 16 receives the message, and optionally adds additional questions, and displays the questionnaire to the patient, who answers it.

Then, the app on the patient communication device 16 transmits the answers back to the remote network service 40. There, the answers are either presented directly to the physician who analyses them and creates a new program. Alternatively, the answers are automatically analyzed by the remote network service 40 and a program is suggested to the physician.

In both cases, the physician transmits the program via remote programming to the implantable medical device 10a. Alternatively, this process can also be triggered without any event but recurring at predefined times and/or in predefined intervals. Both, the alternative and the main procedure can co-exist and be active at the same time. The execution of both is configurable.

The at least one predetermined condition may be fulfilled, if at least one of the plurality of medical parameters P deviates from a predetermined normal range R. Subsequently, the patient information request 12 may be triggered, wherein a type of patient information request 12 triggered depends on which of the at least one of the plurality of medical parameters P deviates from the predetermined normal range R.

Triggering the patient information request 12 comprises sending the patient information request 12, in particular a patient questionnaire, from the server 14 of an IT service provider to the patient communication device 16, in particular a smart phone and/or tablet computing device. The patient information request 12 is moreover accessible via a first front-end application 18 on the patient communication device 16.

Alternatively, instead of sending the patient information request 12 itself, triggering the patient information request 12 can comprise sending a command 20 from a server 14 of an IT service provider to a patient communication device 16, in particular a smart phone and/or tablet computing device, to access the patient information request 12, in particular a patient questionnaire.

The patient medical data D1 and/or the received patient medication data D2 is received by a remote network service 40, in particular operating on the server 14 of the IT service provider. Moreover, the patient information request 12 is sent to an app running on the patient communication device 16 instantly or with a predetermined time delay.

Furthermore, the app running on the patient communication device 16 compares a timestamp of the patient medical data D1, in particular a timestamp of an event triggering the patient information request 12, said timestamp being comprised by the patient information request 12, with calendar data of the patient communication device 16. If a calendar entry is identified which matches and/or overlaps with the timestamp, a first predefined action is taken.

In addition, the calendar entry is at least one of a sports activity, a meeting, a doctor's appointment and sleep. The first predefined action is to add a further information request, in particular a question, to the patient information request 12, said further information request requesting whether the calendar entry matching and/or overlapping with the timestamp has had a physiological effect on the patient, in particular whether the calendar entry may be related to the event triggering the patient information request 12.

The app running on the patient communication device 16 further compares a timestamp of the patient medical data D1, in particular a timestamp of an event triggering the patient information request 12, said timestamp being comprised by the patient information request 12, with physiological data acquired by the wearable medical device 10b, said wearable medical device 10b being at least one of a smart watch and a fitness tracker, wherein if the physiological data acquired by the wearable medical device 10b deviates from a predefined normal range at the time of the timestamp, a second predefined action is taken.

In addition, the second predefined action is to add a further information request, in particular a question, to the patient information request 12, said further information request requesting whether the physiological data acquired by the wearable medical device 10b deviating from the predefined normal range at the time of the timestamp has had a physiological effect on the patient, in particular whether said physiological data may be related to the event triggering the patient information request 12. The patient can thus provide information on a likelihood of the physiological data acquired by the wearable medical device 10b being related to the event.

If the at least one of the plurality of medical parameters P deviates from the predetermined normal range R, an alarm 22 and/or a notification 24 to a health care provider may be triggered. Said alarm 22 and/or a notification 24 to a health care provider is accessible via a second front-end application 26 on a communication device 28, in particular a smart phone and/or a personal computer, of the health care provider.

The normal range R of the plurality of medical parameters P may comprise a predetermined buffer range 30 at an upper and/or lower bound of said normal range R. The alarm 22 is maintained until the at least one of the plurality of medical parameters P deviating from the predetermined normal range R is within the normal range R outside of the buffer range 30.

The algorithm A evaluates a response 32 to the patient information request 12, wherein based on the response 32 an alarm 22 status is maintained or adjusted and/or a notification 24 to the health care provider is generated comprising a recommended action.

If the response 32 to the patient information request 12 indicates that the patient is symptom free (or no symptoms could be captured), a further patient information request 12 is triggered after a predetermined time period T1.

If the alarm 22 is still active after a predetermined time period T1 after triggering the patient information request 12, a further patient information request 12 is triggered after a further predetermined time period T2.

The patient information request 12 is related to an individual patient medical condition, wherein the type of patient information request 12 triggered depends on a type of implant and/or wearable medical device of the patient, the medical data DI comprising the plurality of medical parameters P acquired by the implant and/or wearable medical device of the patient and/or an electronic health record 34 of the patient.

The algorithm A outputs a health score SC, in particular a numerical value, of the patient. The health score SC may incorporate the patient medical data DI comprising the plurality of medical parameters P acquired by the implantable medical device 10a and/or wearable medical device 10b having a first weight W1 and the response 32 to the patient information request 12 having a second weight W2.

The first weight W1 and/or the second weight W2 is adjusted based on the response 32 to the patient information request 12.

If no response 32 to the patient information request 12 is received within a predetermined time period T1, a notification 24 to the health care provider and/or a further patient information request 12 is triggered.

FIG. 2 shows a diagram of a system 1 for providing and/or assessing a patient's medical condition according to the preferred embodiment of the invention.

The system 1 comprises a data storage device 36 configured to receive patient medical data D1 comprising a plurality of medical parameters P acquired by an implantable medical device 10a and/or wearable medical device 10b and/or configured to receive patient medication data D2.

Moreover, the system 1 comprises a computing device 38 configured to apply an algorithm A to the received patient medical data DI and/or the received patient medication data D2. If at least one of the plurality of medical parameters P deviates from a predetermined normal range R, the computing device 38 is further configured to trigger a patient information request 12. A type of patient information request 12 triggered depends on which of the at least one of the plurality of medical parameters P deviates from the predetermined normal range R.

Although specific embodiments have been illustrated and described herein, it will be understood by those skilled in the art that a variety of alternative and/or equivalent implementations exist. It should be noted that the exemplary embodiment or exemplary embodiments are examples only and are not intended to limit the scope, applicability, or configuration in any way.

Rather, the foregoing summary and detailed description provides those skilled in the art with convenient guidance for implementing at least one exemplary embodiment, it being understood that various changes in the functional scope and arrangement of the elements may be made without departing from the scope of the appended claims and their legal equivalents.

In general, this application intends to cover modifications or adaptations or variations of the embodiments disclosed herein. For example, an order of the method steps may be modified. The method may further be carried out sequentially or in parallel, at least in part.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### REFERENCE SIGNS

1 system
10a implantable medical device
10b wearable medical device
12 patient information request
14 server
16 patient communication device
18 first front-end application
20 command
22 alarm
24 notification to health care provider
26 second front-end application
28 communication device
30 buffer range
32 response
34 electronic health record
36 data storage device
38 computing device
40 remote network service
A algorithm
D1 medical data
D2 medication data
P medical parameters
R normal range
SC health score
S1a,b-S3 method steps
T1 predetermined time period
T2 further predetermined time period
W1 first weight
W2 second weight

1. Computer-implemented method for providing and/or assessing a patient's medical condition, comprising the steps of:
receiving patient medical data comprising a plurality of medical parameters acquired by an implantable medical device and/or wearable medical device and/or receiving patient medication data;
applying an algorithm to the received patient medical data and/or the received patient medication data, wherein if at least one predetermined condition is fulfilled, triggering a patient information request.

2. Computer-implemented method of claim 1, wherein applying the algorithm to the received patient medical data and/or the received patient medication data comprises, if at least one of the plurality of medical parameters deviates from a predetermined normal range, triggering the patient information request, wherein a type of the patient information request triggered depends on which of the at least one of the plurality of medical parameters deviates from the predetermined normal range.

3. Computer-implemented method of claim 1, wherein triggering the patient information request comprises sending the patient information request, in particular a patient questionnaire, from a server of an IT service provider to a patient communication device, in particular a smart phone and/or tablet computing device, wherein the patient information request is accessible via a first front-end application on the patient communication device.

4. Computer-implemented method of claim 1, wherein triggering the patient information request comprises sending a command from a server of an IT service provider to a patient communication device, in particular a smart phone and/or tablet computing device, to access the patient information request, in particular a patient questionnaire.

5. Computer-implemented method of claim 1, wherein the patient medical data and/or the received patient medication data is received by a remote network service in particular operating on the server of the IT service provider, and wherein the patient information request is sent to an app running on the patient communication device instantly or with a predetermined time delay.

6. Computer-implemented method of claim 1, wherein the app running on the patient communication device compares a timestamp of the patient medical data, in particular a timestamp of an event triggering the patient information request, said timestamp being comprised by the patient information request, with calendar data of the patient communication device, wherein if a calendar entry is identified which matches and/or overlaps with the timestamp, a first predefined action is taken.

7. Computer-implemented method of claim 6, wherein the calendar entry is at least one of a sports activity, a meeting, a doctor's appointment and sleep, wherein the first predefined action is to add a further information request, in particular a question, to the patient information request, said further information request requesting whether the calendar entry matching and/or overlapping with the timestamp has had a physiological effect on the patient, in particular whether the calendar entry may be related to the event triggering the patient information request.

8. Computer-implemented method of claim 1, wherein the app running on the patient communication device compares a timestamp of the patient medical data, in particular a timestamp of an event triggering the patient information request said timestamp being comprised by the patient information request, with physiological data acquired by the wearable medical device said wearable medical device being at least one of a smart watch and a fitness tracker, wherein if the physiological data acquired by the wearable medical device deviates from a predefined normal range at the time of the timestamp, a second predefined action is taken.

9. Computer-implemented method of claim 8, wherein the second predefined action is to add a further information request, in particular a question, to the patient information request, said further information request requesting whether the physiological data acquired by the wearable medical device deviating from the predefined normal range at the time of the timestamp has had a physiological effect on the patient, in particular whether said physiological data may be related to the event triggering the patient information request.

10. Computer-implemented method of claim 1, wherein the patient information request is related to an individual patient medical condition, wherein the type of patient information request triggered depends on a type of implantable medical device and/or wearable medical device of the patient, the patient medical data comprising the plurality of medical parameters acquired by the medical device and/or wearable medical device of the patient, the received patient medication data-and/or an electronic health record of the patient.

11. Computer-implemented method of claim 1, wherein the algorithm outputs a health score, in particular a numerical value, of the patient, wherein the health score incorporates the patient medical data A-comprising the plurality of medical parameters acquired by the implantable medical device and/or wearable medical device and/or the received patient medication data having a first weight and the response to the patient information request having a second weight.

12. Computer-implemented method of claim 11, wherein the first weight and/or the second weight is adjusted based on the response to the patient information request.

13. System for providing and/or assessing a patient's medical condition, comprising:
a data storage device configured to receive patient medical data comprising a plurality of medical parameters acquired by an implantable medical device and/or wearable medical device and/or configured to receive patient medication data and
a computing device configured to apply an algorithm the received patient medical data-and/or the received patient medication data, wherein if at least one predetermined condition is fulfilled, the computing device is further configured to trigger a patient information request

14. Computer program with program code to perform the method of claim 1 when the computer program is executed on a computer.

15. Computer-readable data carrier containing program code of a computer program for performing the method of claim 1 when the computer program is executed on a computer.
